(19) United States
(12) Patent Application Publication
(10) Pub. No.: US 2024/0290499 A1
(43) Pub. Date: Aug. 29, 2024

(54) Method and System for Predicting Medical Diagnoses Using Machine Learning without Patient Intervention

(71) Applicant: Elevance Health, Inc., Indianapolis, IN (US)

(72) Inventors: Eugene Hsu, Burlingame, CA (US); Jessica Feeney, Chaddsford, PA (US); Joon-Ku Im, Evanston, IL (US); Keea Taylor, Swartz Creek, MI (US); Haoyun Feng, Rye Brook, NY (US); Anthony Nguyen, Pacific Palisades, CA (US); Ayush Mathur, Novi, MI (US); Harsha Arcot, Atlanta, GA (US); Shawn Wang, Kildeer, IL (US)

(21) Appl. No.: 18/587,691

(22) Filed: Feb. 26, 2024

Related U.S. Application Data
(60) Provisional application No. 63/486,861, filed on Feb. 24, 2023.

(51) Int. Cl. G16H 50/30; G16H 10/20; G16H 50/20

(52) U.S. Cl. CPC G16H 50/30; G16H 10/20; G16H 50/20

(57) ABSTRACT

A system and method of predicting a medical diagnosis is disclosed. The method includes receiving claims data, clinical data and demographic data and detecting a prediction target for the diagnosis. If present, inputting the prediction indicator and the clinical data into a machine learning model to predict diagnosis risk, to create a diagnosis risk score; determining a care seeking propensity score, from the demographic data; weighting the diagnosis risk score by the care seeking propensity score to create a weighted diagnosis risk score; determining whether the weighted diagnosis risk score indicates a likelihood of the medical diagnosis; and, in response, transmitting a recommendation for further evaluation. The machine learning model may be trained using historical claims data, clinical data, and demographic data and may be trained to detect correlation between medical diagnosis signals identified from the training data, and a positive result from a screening mechanism the medical diagnosis.

### CROSS REFERENCE TO RELATED APPLICATIONS

This application claims the benefit of U.S. Provisional Patent Application No. 63/486,861, filed Feb. 24, 2023, the disclosure of which is incorporated herein by reference in its entirety.

### TECHNICAL FIELD

The disclosed implementations relate generally to medical screening, and relate more specifically to building and using machine learning models to pre-screen patients for certain health conditions, including mental health conditions, by analyzing data relating to the patient, independent of or as an enhancement to interviewing or examining the patient.

### BACKGROUND

As is currently known in the art, the definition and detection of many mental health disorders and risk factors, including depression, is driven, at least as an initial step, through a clinically accepted questionnaire. One such questionnaire, whose results are accepted as the industry standard for diagnosis of depression and other mental health disorders, is the Patient Health Questionnaire 2 (“PHQ-2”). Historically, human to human delivery of the questionnaire was a primary driver in identification of depression and other mental health disorders and risk factors. However, this may be time intensive, expensive, and suffer from selection bias in that certain populations are unlikely to get screened despite an increased likelihood of a positive result based on the screening. Identification of depression and other mental health disorders has previously relied on specially trained personnel administering a questionnaire often in a setting requiring dedicated appointment time to have a human-to-human interaction by phone or in-person. However, identifying people who should receive the questionnaire in the first place suffers from information asymmetries. Certain risk factors, which may be known in the aggregate, may not be known to be present in any particular patient, either by potential patients themselves nor by mental health specialists. Other health care providers, who may know of the existence of the risk factors, may not know that they are risk factors, as mental health may not be their specialty.

Technical challenges to accurately predicting the existence of depression and other mental health disorders and risk factors for a specific individual within a defined subpopulation have included determining which datasets reliably drive a prediction of a mental health disorder or a risk factor for a mental health disorder, such as clinical, social, sociodemographic, etc., and obtaining the data in a timely manner. Conventionally, this data is most commonly acquired via individual interviews with patients. A critical mass of data, sufficient to power a robust and accurate model, was either not systemically available, or cost prohibitive to acquire, or, in some instances, both. Data sets needed to reliably drive a prediction of depression and other mental health disorders, or risk factors of the same, were not reliable or systemically consistent.

Modern data structures, electronic medical records and other types of data sharing across the healthcare ecosystem, and industry standards, have enabled a greater consistency and accuracy of the respective data. However, leveraging the obtained data sets in a way that reliably predicts the existence of depression and other mental health disorders and risk factors remained difficult. An automated process, that selects patients for further screening, based on risk factors detected in vast amounts of accumulated data relating to the patient that cannot be reviewed manually in an efficient manner, would be advantageous, allowing for heretofore undiagnosed individuals suffering from depression to receive diagnoses and treatment.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method of predicting a medical diagnosis for a patient. The method of predicting also includes receiving claims data, clinical data and demographic data relating to the patient. The method also includes determining, from the claims data whether a prediction target for the medical diagnosis is present. The method also includes in response to a determination that the prediction target is present: inputting the prediction indicator and the clinical data into a machine learning model to predict diagnosis risk, to create a diagnosis risk score; determining a care seeking propensity score, from the demographic data, where the care seeking propensity score is related to whether the patient is a member of a group with a propensity to seek care that is lower than a reference care seeking propensity score for other patients; weighting the diagnosis risk score by the care seeking propensity score to create a weighted diagnosis risk score; determining whether the weighted diagnosis risk score indicates a likelihood of the medical diagnosis; and in response to the determination that the weighted diagnosis risk score indicates a likelihood of the medical diagnosis, transmitting a recommendation for further evaluation to a digital device associated with the patient. The machine learning model may be trained using training data may include historical claims data, historical clinical data, and historical demographic data, from a population of prior patients. The machine learning model may be trained to detect correlation between medical diagnosis signals identified from the training data, and a positive result from a screening mechanism for likelihood of the medical diagnosis. Other embodiments of this aspect include corresponding computer systems, apparatus, and computer programs recorded on one or more computer storage devices, each configured to perform the actions of the methods.

Implementations may include one or more of the following features. The prediction target may be a claim for a medical procedure within a predetermined amount of time into the future, and the medical procedure may be unrelated to the medical diagnosis. The prediction target may be a claim for an upcoming surgical procedure. The medical diagnosis may be a mental health diagnosis. The screening mechanism may be a questionnaire. The method may include the steps of, after the determination that the weighted diagnosis risk score indicates a likelihood of the medical diagnosis, performing the traditional screening mechanism on the patient to obtain a screening result, and enhancing the training of the machine learning model using the screening result, and using at least one of the claims data, the clinical data, and the demographic data. The method may include the steps of obtaining a vocal sample of the patient; performing vocal sentiment analysis on the vocal sample; and including vocal sentiment data in the social data. The demographic data may include data relating to a location where the patient resides. The clinical data may include comorbidity data. The clinical data may include medication history. The clinical data may include data relating to a history of usage of health services by the patient. The clinical data may include data relating to at least one of patient age, patient insurance coverage, distance between a residence of the patient and medical care, patient access to transportation, patient access to food, and patient access to shelter. The clinical data may include data relating to at least one of health conditions, use of specific medications, medical visits, hospitalizations, laboratory tests, vaccination status, sleep studies, and drug use. The clinical data may be derived at least in part from an electronic medical record relating to the patient. Implementations of the described techniques may include hardware, a method or process, or computer software on a computer-accessible medium.

One general aspect includes a system for predicting a medical diagnosis for a patient, independent of interviewing or examining the patient, the system comprising a processor configured to: receive claims data, clinical data and demographic data relating to the patient. The processor is also configured to determine, from the claims data whether a prediction target for the medical diagnosis is present. The processor is also configured to, in response to a determination that the prediction target is present: input the prediction indicator and the clinical data into a machine learning model to predict diagnosis risk, to create a diagnosis risk score; determine a care seeking propensity score, from the demographic data, where the care seeking propensity score is related to whether the patient is a member of a group with a propensity to seek care that is lower than a reference care seeking propensity score for other patients; weight the diagnosis risk score by the care seeking propensity score to create a weighted diagnosis risk score; determine whether the weighted diagnosis risk score indicates a likelihood of the medical diagnosis; and in response to the determination that the weighted diagnosis risk score indicates a likelihood of the medical diagnosis, transmit a recommendation for further evaluation to a digital device associated with the patient. The machine learning model may be trained using training data may include historical claims data, historical clinical data, and historical demographic data, from a population of prior patients. The machine learning model may be trained to detect correlation between medical diagnosis signals identified from the training data, and a positive result from a screening mechanism for likelihood of the medical diagnosis. Other embodiments of this aspect include corresponding computer systems, apparatus, and computer programs recorded on one or more computer storage devices, each configured to perform the actions of the methods.

Implementations may include one or more of the following features. The prediction target may be a claim for a medical procedure within a predetermined amount of time into the future, and the medical procedure may be unrelated to the medical diagnosis. The prediction target may be a claim for an upcoming surgical procedure. The medical diagnosis may be a mental health diagnosis. The screening mechanism may be a questionnaire. The processor is also configured to, after the determination that the weighted diagnosis risk score indicates a likelihood of the medical diagnosis, perform the traditional screening mechanism on the patient to obtain a screening result, and enhance the training of the machine learning model using the screening result, and using at least one of the claims data, the clinical data, and the demographic data. The processor is also configured to obtain a vocal sample of the patient; perform vocal sentiment analysis on the vocal sample; and include vocal sentiment data in the social data. The demographic data may include data relating to a location where the patient resides. The clinical data may include comorbidity data. The clinical data may include medication history. The clinical data may include data relating to a history of usage of health services by the patient. The clinical data may include data relating to at least one of patient age, patient insurance coverage, distance between a residence of the patient and medical care, patient access to transportation, patient access to food, and patient access to shelter. The clinical data may include data relating to at least one of health conditions, use of specific medications, medical visits, hospitalizations, laboratory tests, vaccination status, sleep studies, and drug use. The clinical data may be derived at least in part from an electronic medical record relating to the patient. Implementations of the described techniques may include hardware, a method or process, or computer software on a computer-accessible medium.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is an overview of a system that may be used to perform a depression prediction algorithm in accordance with one embodiment of the present disclosure.

FIG. 2 is a flow diagram of a process for identifying patients for depression screening in accordance with one embodiment of the present disclosure.

FIG. 3 is a flow diagram representing a process for repetitive implementation of the process of FIG. 2.

FIGS. 4A and 4B are a flow diagram of a method in accordance with one embodiment of the present disclosure.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

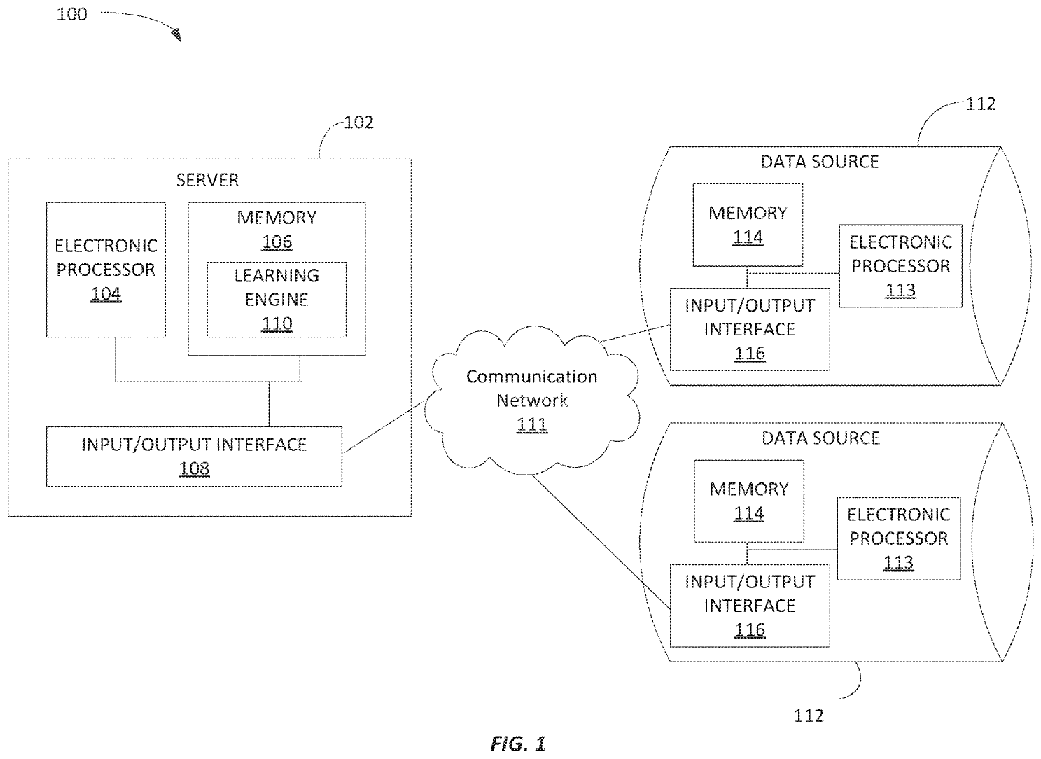

FIG. 1 illustrates a system 100 for performing machine learning to predict a medical diagnosis for a patient, independent of interviewing or examining the patient. according to some embodiments of the invention. The system 100 includes a server 102 that includes a plurality of electrical and electronic components that provide power, operational control, and protection of the components within the server 102. For example, as illustrated in FIG. 1, the server 102 may include an electronic processor 104 (e.g., a microprocessor, application-specific integrated circuit (ASIC), or another suitable electronic device), a memory 106 (e.g., a non-transitory, computer-readable storage medium), and an input/output interface 108. The electronic processor 104, the memory 106, and the input/output interface 108 communicate over one or more connections or buses. The server 102 illustrated in FIG. 1 represents one example of a server and embodiments described herein may include a server with additional, fewer, or different components than the server 102 illustrated in FIG. 1. Also, in some embodiments, the server 102 performs functionality in addition to the functionality described herein. Similarly, the functionality performed by the server 102 (i.e., through execution of instructions by the electronic processor 104) may be distributed among multiple servers. Accordingly, functionality described herein as being performed by the electronic processor 104 may be performed by one or more electronic processors included in the server 102, external to the server 102, or a combination thereof.

The memory 106 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof. The electronic processor 104 executes computer-readable instructions (“software”) stored in the memory 106. The software may include firmware, one or more applications, program data, filters, rules, one or more program modules, and other executable instructions. For example, the software may include instructions and associated data for performing the methods described herein. For example, as illustrated in FIG. 1, the memory 106 may store a learning engine 110 (i.e., software) for performing data analytics for prediction of one or more mental health conditions as described herein (e.g., processing training information to develop models). However, in other embodiments, the functionality described herein as being performed by the learning engine 110 may be performed through one or more software modules stored in the memory 106 or external memory.

The input/output interface 108 allows the server 102 to communicate with devices external to the server 102. For example, as illustrated in FIG. 1, the server 102 may communicate with one or more data sources 112 through the input/output interface 108. In particular, the input/output interface 108 may include a port for receiving a wired connection to an external device (e.g., a universal serial bus (“USB”) cable and the like), a transceiver for establishing a wireless connection to an external device (e.g., over one or more communication networks 111, such as the Internet, a local area network (“LAN”), a wide area network (“WAN”), and the like), or a combination thereof.

In some embodiments, the server 102 also receives input from one or more peripheral devices, such as a keyboard, a pointing device (e.g., a mouse), buttons on a touch screen, a scroll ball, mechanical buttons, and the like through the input/output interface 108. Similarly, in some embodiments, the server 102 provides output to one or more peripheral devices, such as a display device (e.g., a liquid crystal display (“LCD”), a touch screen, and the like), a printer, a speaker, and the like through the input/output interface 108. In some embodiments, output may be provided within a graphical user interface (“GUI”) (e.g., generated by the electronic processor 104 executing instructions and data stored in the memory 106 and presented on a touch screen or other display) that enables a user to interact with the server 102. In other embodiments, a user may interact with the server 102 through one or more intermediary devices, such as a personal computing device laptop, desktop, tablet, smart phone, smart watch or other wearable, smart television, and the like). For example, a user may configure functionality performed by the server 102 as described herein by providing data to an intermediary device that communicates with the server 102. In particular, a user may use a browser application executed by an intermediary device to access a web page that receives input from and provides output to the user for configuring the functionality performed by the server 102.

As illustrated in FIG. 1, the system 100 includes one or more data sources 112. Each data source 112 may include a plurality of electrical and electronic components that provide power, operational control, and protection of the components within the data source 112. In some embodiments, each data source 112 represents a server, a database, a personal computing device, or a combination thereof. For example, as illustrated in FIG. 1, each data source 112 may include an electronic processor 113 (e.g., a microprocessor, ASIC, or other suitable electronic device), a memory 114 (e.g., a non-transitory, computer-readable storage medium), and an input/output interface 116. The data sources 112 illustrated in FIG. 1 represents one example of data sources and embodiments described herein may include a data source with additional, fewer, or different components than the data sources 112 illustrated in FIG. 1. Also, in some embodiments, the server 102 communicates with more or fewer data sources 112 than illustrated in FIG. 1.

The input/output interface 116 allows the data source 112 to communicate with external devices, such as the server 102. For example, as illustrated in FIG. 1, the input/output interface 116 may include a transceiver for establishing a wireless connection to the server 102 or other devices through the communication network 111 described above. Alternatively or in addition, the input/output interface 116 may include a port for receiving a wired connection to the server 102 or other devices. Furthermore, in some embodiments, the data sources 112 also communicate with one or more peripheral devices through the input/output interface 116 for receiving input from a user, providing output to a user, or a combination thereof. In other embodiments, one or more of the data sources 112 may communicate with the server 102 through one or more intermediary devices. Also, in some embodiments, one or more of the data sources 112 may be included in the server 102.

The memory 114 of each data source 112 may store medical data, claims data, demographic data, and the like. For example, the data sources 112 may include an electronic medical record (“EMR”) database, a claims database, and the like. In some embodiments, as noted above, data stored in the data sources 112 or a portion thereof may be stored locally on the server 102 (e.g., in the memory 106).

When a patient is scheduled for or otherwise due to undergo certain medical procedures not relating to mental health, the future occurrence of the procedure itself may exacerbate an underlying depression and/or other mental health disorders and risk factors. However, providers who are scheduled to perform such procedures that may be triggers for mental health are busy and are often more focused on caring for the patient's physical health. Also, they generally do not specialize in mental health, and may therefore omit important social and mental health screenings during patient encounters. For example, it is believed that more than 20% of seniors over the age of 65 have clinical depression or other mental health disorders or risk factors. However, the number that were identified through healthcare claims data from a national insurer with a claims dataset representing over 46 million subscribers, was only a small fraction of that 20%. Accordingly, a digital-first approach is herein disclosed, for building an adaptive artificial intelligence algorithm. Such an intelligence algorithm may be trained to use data that may be in the possession of or accessible by a medical insurer that covers, and therefore has access to data relating to, a wide population. Data of comparable scope and breadth may also be available from other sources. Such data may include clinical, social, claims, and sociodemographic data. The algorithm herein disclosed may use the data, herein discussed, to predict positive results of a PHQ-2 screening, for depressive symptoms, or other diagnostic tools for other mental health conditions.

Previous attempts at using machine learning algorithms to make population-wide predictions, for results of screening questionnaires for depression and other mental health disorders, have had limited success, in large part because the data used for training the machine learning algorithm has been insufficient to the task. Some previous attempts may have used only electronic medical record (“EMR”) data, and/or self-reported textual or social media data. Models using traditional claims data, have been explored to understand the ability to predict the existence of depression and other mental health disorders and risk factors with traditional payor data. Traditional payor data, especially when considered alone, has also not proven to have clinical value. Analysis using traditional payor data was also not rooted in a clinically accepted definition of depression, nor of other mental health disorders and risk factors. Traditional payor data has been analyzed only to the extent the payor data related to the mental health condition explicitly, e.g., if depression had been previously diagnosed or detected.

PHQ-2 may, by way of example, ask the following questions:

Over the last 2 weeks, how often have you
been bothered by any of the following problems:
Little interest or pleasure in doing things

| | |
|---|---|
| Not at all | 0 |
| Several days | 1 |
| More than half the days | 2 |
| Nearly every day | 3 |

Feeling down, depressed, or hopeless

| | |
|---|---|
| Not at all | 0 |
| Several days | 1 |
| More than half the days | 2 |
| Nearly every day | 3 |

Another questionnaire, Social Determinants of Health (“SDoH”) may be used. SDoH may ask questions relating to financial strain, food, shelter, and transportation. For financial strain, SDoH may ask “how hard is it for you to pay for the very basics like food, housing, medical care and heating.” SDoH may then offer three options for how difficult the patient finds it to pay for basics, and may also offer a fourth answer such as “choose not to answer.” SDoH may then assign a numerical value, or score, to each answer.

Similarly, SDoH may ask a patient “Within the past 12 months, the food you bought just didn't last and you didn't have money to get more?” SDOH may offer three answers, e.g., “sometimes,” “often” and “never,” and may also offer a “choose not to answer” option, and assign a score for each. SDoH may also ask a patient whether he or she has a steady place to live, and/or access to reliable transportation, and score the results. SDoH may ask all four of these questions, and then, based on the individual answers and/or a sum of the answers, may refer the patient to treatment.

A loneliness questionnaire, such as the UCLA 3-Item Loneliness Scale, may also be used. A loneliness questionnaire may include questions such as “how often do you feel that you lack companionship.” “how often do you feel left out,” and “how often do you feel isolated from others.” A loneliness questionnaire may offer multiple choice answers (e.g., “often.” “sometimes,” or “hardly ever”) to these questions, and may also score the results and evaluate the individual scores or a sum of them.

Initial screening may be improved by a closed loop, adaptive algorithm that predicts positive PHQ-2 and other clinical behavioral health screening questionnaire results. as disclosed herein. Such an adaptive algorithm may utilize and/or be trained upon categories of data that may be shown to, or learned by the algorithm to, include a correlation with a positive diagnosis of depression or other mental health conditions, diagnoses or issues.

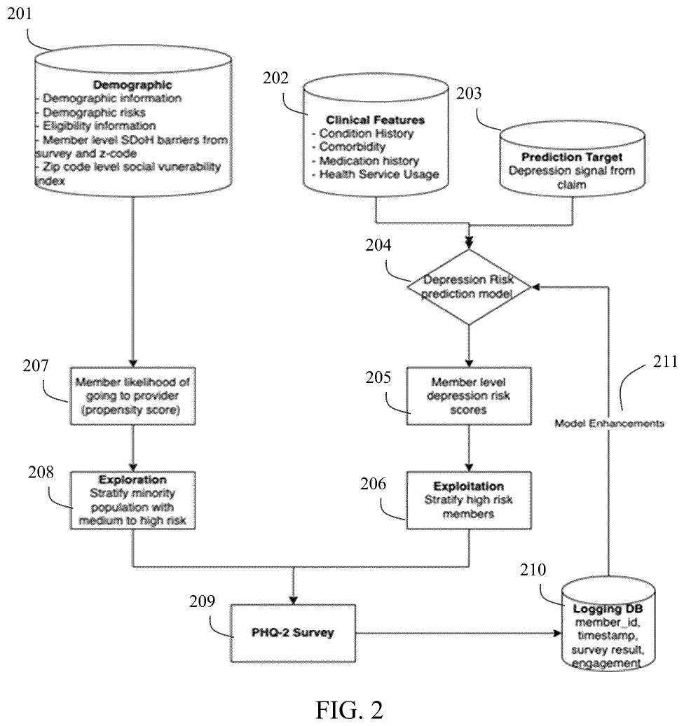

Turning now to FIG. 2, an information flow diagram of an adaptive algorithm for depression screening in accordance with some embodiments of the present disclosure, is shown. An adaptive algorithm may use demographic data 201, which may include SDoH data. Demographic data 201 may include demographic information such as race, gender, age, etc. Demographic data 201 may also include data relating to demographic risk factors specific to the mental health condition being diagnosed, in this case, depression. Demographic data 201 may also include insurance eligibility information, which may include information relating to a length of time the patient has had medical insurance, e.g., with the provider or in general, and may also include information relating to features of the insurance carried by the patient. Demographic data 201 may also SDoH data, which may include Z-codes, which are recognized markers that indicate that a patient is experiencing a symptom that merits treatment but that, at least in current practice, are not indicative of a specific diagnosis. SDoH data may also barrier data, which may be derived from surveys completed by the patient, and may include usage of immediate assistance services such as “Aunt Bertha”, which is a directory of assistance services for urgent needs such as food, shelter and transportation. Demographic data 201 may also include ZIP code level social vulnerability data, which may include correlations between depression and where the patient lives.

In some embodiments, specific demographic data used may include Age, Insurance Coverage, Distance to Medical Care (e.g., to ER/Urgent Care), Lack of food, Lack of shelter, and Lack of transportation.

An adaptive algorithm may also include clinical features 202. Clinical features 202 may include the patient's condition history, which may include previous treatments for substance abuse. Clinical features 202 may also include diagnosis of comorbidities in general, or specific comorbidities that may be determined, e.g., by the algorithm itself, to correlate with mental health conditions. Clinical features 202 may also include prescriptions for medications that may be known or later identified to correlate with one or more mental health conditions. Clinical features 202 may also include the patient's history of health service usage, which may also include laboratory results, including but not limited to specific blood or urine analysis results, that may also be known or later identified to correlate with one or more mental health conditions.

In some embodiments, clinical features that are may be correlated with an increased risk of depression may include: Prior Behavioral Health Conditions (Anxiety, Mood, Depression and other mental health disorders and risk factors, Substance Abuse, Schizophrenia, Alcohol use), Prior Physical Health Conditions (Nervous System, Insomnia, Respiratory, Hypertension, Antidepressant use, Serotonin Reuptake Inhibitors, Opioid Use, Benzodiazepine Use, Anticonvulsant Use, Use of Antianxiety medications, Inpatient, Readmittance, and Emergency Room Visits, Evaluation and Management Usage, Other Outpatient usage, Other medication usage, Radiology usage, Vaccination usage, Laboratory usage, Mortality score, Sleep studies and Drug screening.

Demographic data such as age, gender, proximity to health care resources 201, clinical features such as outpatient and specialty care utilization, prior diagnosis, prescription usage 202, and prediction target 203 may each comprise a plurality of signals that may from time to time be determined to correlate with the output of a machine learning model designed to detect, e.g., depression or other mental illnesses.

Prediction target 203 may include claims data, which may include data relating to health insurance claims for physical and behavioral health issues that may signal a propensity for depression. Claims data may, in some embodiments, relate to a recent claim for an upcoming procedure that may increase the risk of depression in patients who are scheduled to undergo the specific procedure.

In some embodiments, the below may reflect notification time in advance of claims for certain medical procedures that may be found to increase risk of depression.

| Surgery Type | Average | Minimum | Maximum |
|---|---|---|---|
| Auditory | 19 | 0 | 69 |
| Cardiovascular | 12 | 0 | 83 |
| Digestive System | 15 | 0 | 132 |
| Digestive System - Bariatric | 44 | 22 | 61 |
| Digestive System - | 47 | 5 | 117 |
| Laparoscopic | | | |
| Endocrine | 13 | 0 | 39 |
| Eye and Ocular Adnexa | 12 | 0 | 76 |
| Female Genital | 16 | 0 | 109 |
| General Surgical | 2 | 0 | 12 |
| Hemic and Lymphatic Systems | 13 | 0 | 64 |
| Integumentary System | 14 | 0 | 118 |
| Intersex | 34 | 34 | 34 |
| Male Genital System | 15 | 0 | 69 |
| Maternity Care Delivery | 10 | 0 | 28 |
| Mediastinum and Diaphragm | 14 | 2 | 31 |
| Musculoskeletal System | 13 | 0 | 105 |
| Nervous | 15 | 0 | 63 |
| Operating microscope | 16 | 10 | 21 |
| procedures | | | |
| Reproductive | 14 | 0 | 27 |
| Respiratory System | 14 | 0 | 110 |
| Urinary System | 14 | 0 | 143 |
| Grand Total | 14 | 0 | 143 |

Because of the above, authorizations provide a triggering mechanism via prediction target 203 to outreach a member at a time when they are at greater risk of having depression surface. By identifying which of those members are at highest risk for depression, outreach can be made at the time that the member needs it most. Access to a patient and the patient's relevant data, prior to their care episode allows depression risk prediction model 204 to both identify the patient's risk at the particular time in their care journey that the model 204 is run, as well as to outreach to them to facilitate access to care.

In some embodiments, depending on the nature of the entity that is gathering the data and/or executing depression risk prediction model 204, the demographic data 201, clinical features 202, and prediction targets 203, may already be available without further intervention with the patient, allowing depression risk prediction model 204 to identify patients at risk for depression without requiring their specific consent to data gathering or analysis of this type.

Clinical features 202 and prediction target 203 may then be fed to depression risk prediction model 204, which is discussed in more detail below. Depression risk prediction model 204 may include machine learning elements, which may be trained on datasets including similar types of data of that evaluated relating to the individual patient, which may include demographic training data, clinical training data, and claims training data. Initially, depression risk prediction model 204 may be trained based on known risk factors that may appear in demographic data 201, clinical features 202 and/or prediction target 203. Depression risk prediction model 204 may then produce a depression risk score 205. Patients may be stratified into risk tiers 206 or otherwise classified on the basis depression risk score 205.

In some embodiments, depression risk prediction model 204 may utilize multiple models, which may include a base model and a learning model. The base model, in some embodiments may utilize Xboost Early Stopping to predict if a member has a likelihood of developing depression in the next six months. The target of depression risk prediction model 204 is a diagnosis of depression or other mental disorders within the next six months on a claim. Predictions will be based on most recent incurred data available that month. In some embodiments, depression risk prediction model 204 may be deployed using ETL pipeline on AWS snowflake.

Demographic data 201, clinical features 202 and prediction target 203 may in some embodiments be both broad and deep. In some embodiments, such a data set may be broad because it draws from data relating to millions of patients, e.g., of the same insurance carrier. The data set may also be deep because it does not require additional consent and multiple modalities of data can be unified and associated with the same patient, including Aunt Bertha, EMR, and claims data.

In some embodiments, an Initial model summary may be trained on population data similar to the following:

| | Training | OOT Validation |
|---|---|---|
| QP Count | 1,890,474 | 1,803,386 |
| Target Count with QP | 364,104 | 316,295 |
| cut-off date | 2021 May 31 | 2021 Nov. 30 |
| QP period (−24 M) | Jun. 1, 2019-May 31, 2021 | Dec. 1, 2019-Nov. 30, 2021 |
| target period (+6 M) | Jun. 1, 2021-November 2021 | Dec. 1, 2021-May 2022 |

In some embodiments, a care seeking propensity score 207 may be derived from demographic data 201. Care seeking propensity score 207 may be calculated based on demographic data 201, to reflect knowledge, which may be pre-existing or may be later learned by the algorithm, relating to demographic characteristics that may correlate with, and a patient being more or less likely to seek medical care, and/or to seek medical care specifically for mental health. Care seeking propensity score 207 may take the form of a weight that is applied to depression risk score 205. In some embodiments, care seeking propensity scoring and depression risk scoring will be performed through an ENSO platform. In some embodiments, the use of demographic data 201 may be filtered, adjusted or otherwise influenced by an auditing framework designed to reduce or eliminate discrimination and bias within the data modeling. In some embodiments, scoring of care seeking propensity score 207 may be adjusted to reflect the result of such an auditing framework, the model may also be continuously monitored and adjusted for said items. In some embodiments, patients may be stratified (208) by care seeking propensity score 207.

Patients may then be identified to receive a PHQ-2 Survey 209, on the basis of the patient's risk tier 206, as weighted by the patient's care seeking propensity score 207. In some embodiments, the result a patient being selected may include the patient being notified and/or being referred to a mental health professional to administer the PHQ-2 Survey 209.

After the patient has been administered a PHQ-2 survey, the results of the survey 209 may be logged in logging database 210. In some embodiments, the results of the survey may be saved in logging database 210 along with the patient's member ID, a time stamp of when the survey 209 was administered, a result, and/or information relating to whether the patient engaged with treatment if the survey 209 indicated treatment a treatment recommendation. In some embodiments, individual questions from the survey 209 may also be saved in logging database 210. In some embodiments, logging database 210 may include either Hadoop clusters or AWS servers.

Questionnaire data from survey 209, saved in logging database 210, may then be used for model enhancement 211 to enhance depression risk prediction model 204. Depression risk prediction model 204 may be refined to more accurately predict which clinical features 202 or prediction targets 203 may correlate to positive results from the eventual survey 209. Utilizing gathered assessment data, depression risk prediction model 204 may be developed utilizing reinforcement learning through the gathering of real time data based on member feedback, including results of survey 209 and correlation between those results and the demographic, clinical and claims based data associated with the same patient. Features may also be derived based on member's commonalities to further enhance the accuracy of identifying a person at risk of developing depression. The adaptive algorithm may also use questionnaire data from other questionnaires, which may include responses to Health Risk Assessment (“HRA”) questionnaires.

In addition to claims data, SDoH data, medical data, and questionnaire data, an adaptive algorithm as disclosed herein may also be trained to evaluate, or use evaluations of, vocal sentiment analysis relating to the patient being evaluated. Vocal sentiment analysis may be performed on recorded calls between the patient and, e.g., a provider or an insurer. In some embodiments, vocal sentiment analysis may be performed on calls that do not have, as their subject matter, the mental health of the patient. They may instead be calls on an unrelated topic, such as a status of an insurance claim, scheduling of a medical procedure, e.g., one that is not mental health related, or other customer services related conversations, where there is a substantial sample of the patient's voice such that sentiment analysis can be performed without regard to content.

In some embodiments, vocal analysis may be able to predict mental health conditions such as depression or cognitive impairment, including cognitive impairment that may be in early stages or mild. In some embodiments, vocal analysis is tone based, which may be different from textual based sentiment analysis that may use the words and phrases being uttered rather than the tone of the speaker's voice. Vocal analysis may be content agnostic.

Although sufficient vocal samples would be required to perform tone based analysis on a particular patient, in some embodiments, vocal analysis may be language agnostic, such that the tone of voice may be analyzed without regard to what language is being spoken in the content of the speech being analyzed. In some embodiments, vocal analysis may be baseline agnostic, such that it can be performed without having a prior “baseline” result from the same individual.

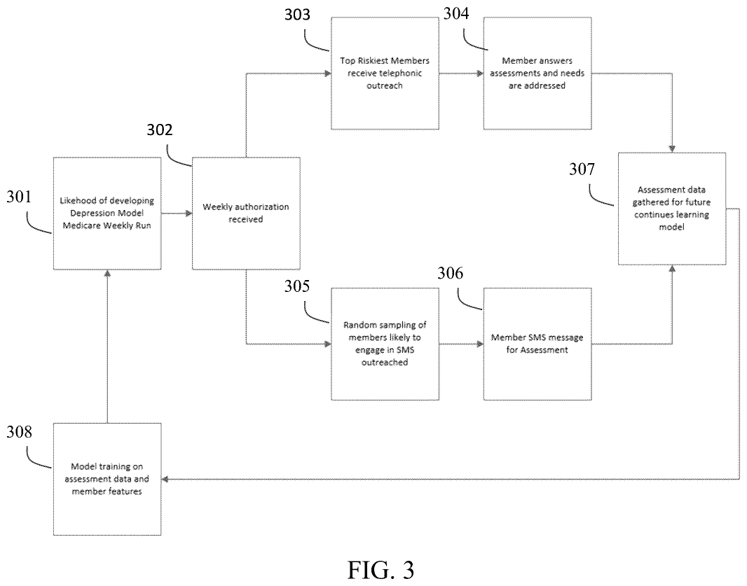

Turning now to FIG. 3, a process flow for repeated implementation of the process of FIG. 2, as well as the actions that may be taken based on the results of the process of FIG. 2, is shown. In some embodiments, the process of FIG. 2 may be run (301) weekly and a weekly authorization may be received (302). A weekly authorization may be or include an authorization to proceed with real world outreach efforts targeted at patients identified by the process of FIG. 2. The Top Riskiest Members after accounting for depression risk score 205 and care seeking propensity score 207 may receive (303) telephonic outreach. The Patient may then receive assessments, e.g., survey 209, and may provide (304) answers to the assessments and then may receive follow up treatment if necessary. Weekly authorization may also include authorization to engage (305) a random sampling of patients, without regard to the results of the process of FIG. 2. These patients may in some embodiments be identified as more likely to be responsive to outreach, and may be given the surveys, for purposes of a “control” group, so that when model enhancement 211 is run, depression risk prediction model 204 may be further trained with data from survey 209 performed on patients who were not identified as having risk factors, in addition to data from patients who were identified as having risk factors. Engagement (305) may be accomplished via SMS message, although other outreach methods may be used. Identified users may then respond (306) to the SMS message and engage with, e.g., survey 209.

Assessment data may then be gathered (307) from both the patients identified with risk factors as a result of the depression risk score 205 and care seeking propensity score 207, and those without risk factors, who were either randomly selected, selected for propensity to respond to outreach, or otherwise selected. Assessment data may in some embodiments be gathered in logging database 210. The model may then be trained (308) on the assessment data, which, in some embodiments, may occur via model enhancement 211 of FIG. 2. Accordingly, the model continues to learn and adjust the correlation between the signals, including those shown in FIG. 3, and positive or negative results in depression screening survey 209.

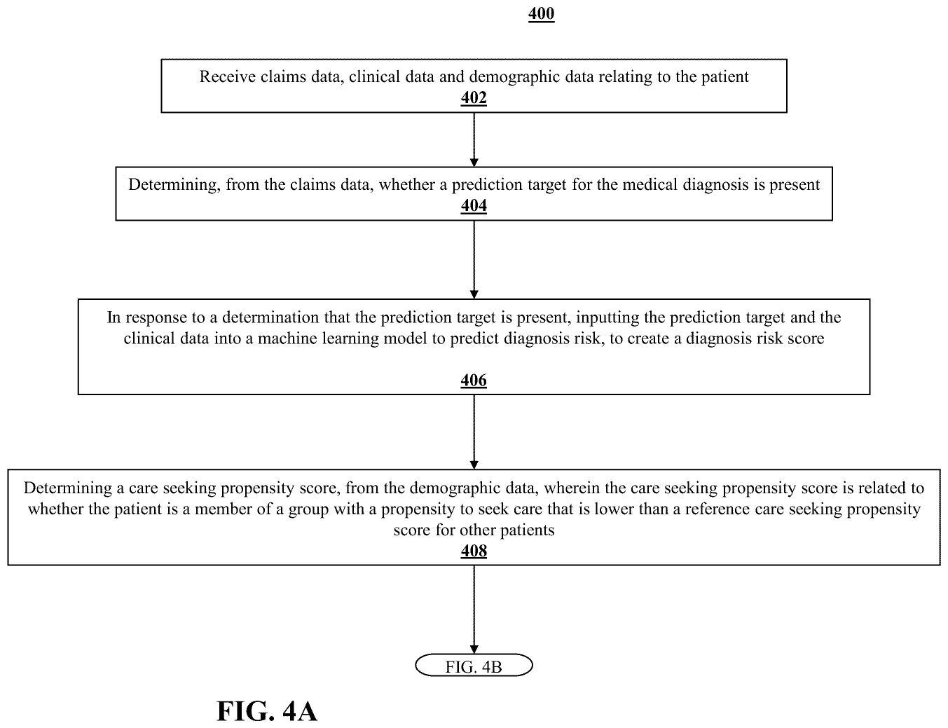

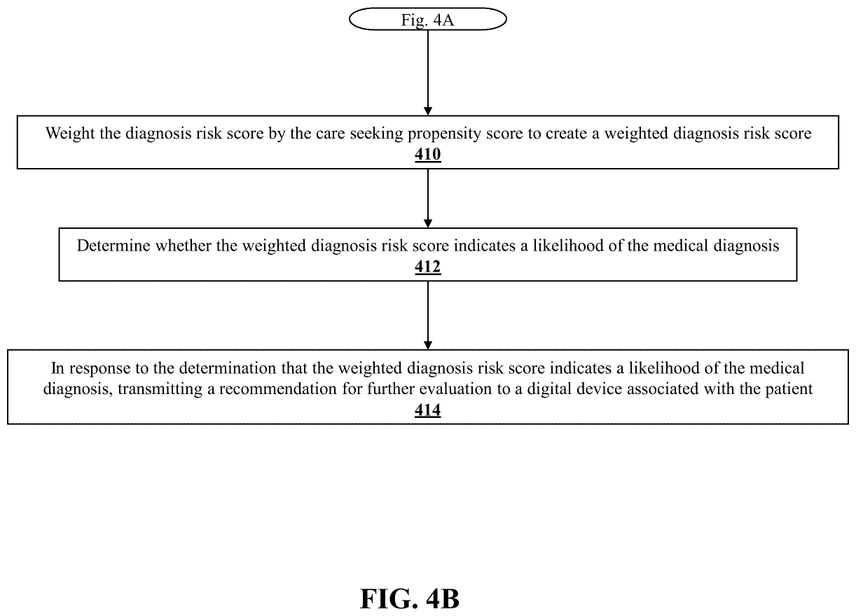

Turning now to FIGS. 4A and 4B, where a flow diagram of a method of predicting a medical diagnosis for a patient, independent of interviewing or examining the patient, in accordance with an embodiment of the present disclosure, is shown. The method includes receiving (402) claims data, clinical data, such as clinical features 202 as discussed with reference to FIG. 2, and demographic data, such as demographic data 201, relating to the patient. The method further includes determining (404), from the claims data, whether a prediction target, such as prediction target 203, for the medical diagnosis is present. The method further includes, in response to a determination that the prediction target is present, inputting (406) the prediction target and the clinical data into a machine learning model, such as depression risk prediction model 204, to predict diagnosis risk, to create a diagnosis risk score, such as depression risk score 205, and determining (408) a care seeking propensity score, such as care seeking propensity score 207, from the demographic data. The care seeking propensity score may be related to whether the patient is a member of a group with a propensity to seek care that is lower than a reference care seeking propensity score for other patients.

The method further includes weighting (410) the diagnosis risk score by the care seeking propensity score to create a weighted diagnosis risk score. The method further includes determining (412) whether the weighted diagnosis risk score indicates a likelihood of the medical diagnosis. The method further includes, in response to the determination that the weighted diagnosis risk score indicates a likelihood of the medical diagnosis, transmitting (414) a recommendation for further evaluation to a digital device associated with the patient. The machine learning model may be trained using training data comprising historical claims data, historical clinical data, and historical demographic data, from a population of prior patients. The machine learning model may also be trained to detect correlation between medical diagnosis signals identified from the training data, and a positive result from a screening mechanism for likelihood of the medical diagnosis.

The prediction target may be a claim for a medical procedure within a predetermined amount of time into the future, and the medical procedure may be unrelated to the medical diagnosis. The prediction target may be a claim for an upcoming surgical procedure. The medical diagnosis may be a mental health diagnosis. The screening mechanism, may be a questionnaire.

The method may also include the steps of, after the determination that the weighted diagnosis risk score indicates a likelihood of the medical diagnosis, performing the screening mechanism on the patient to obtain a screening result, and further training of the machine learning model using the screening result, and using at least one of the claims data, the clinical data, and the demographic data.

The method may also include the steps of obtaining a vocal sample of the patient, performing vocal sentiment analysis on the vocal sample, and including vocal sentiment data in the clinical data.

The demographic data may include data relating to a location where the patient resides. The clinical data may include comorbidity data. The clinical data may include medication history.

The clinical data may also include data relating to a history of usage of health services by the patient.

The clinical data may also include data relating to at least one of patient age, patient insurance coverage, distance between a residence of the patient and medical care, patient access to transportation, patient access to food, and patient access to shelter.

The clinical data may also include data relating to at least one of health conditions, use of specific medications, medical visits, hospitalizations, laboratory tests, vaccination status, sleep studies, and drug use.

The clinical data may be derived at least in part from an electronic medical record relating to the patient.

Persons having skill in the art will realize that depression is an exemplar of a mental health conditions, and the disclosed system and method may be adapted to identify patients with risk factors for other mental health conditions.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

What is claimed is:

1. A method of predicting a medical diagnosis for a patient, independent of interviewing or examining the patient, the method comprising:
receiving claims data, clinical data and demographic data relating to the patient;
determining, from the claims data, whether a prediction target for the medical diagnosis is present;
in response to a determination that the prediction target is present:
inputting the prediction target and the clinical data into a machine learning model to predict diagnosis risk, to create a diagnosis risk score;
determining a care seeking propensity score, from the demographic data, wherein the care seeking propensity score is related to whether the patient is a member of a group with a propensity to seek care that is lower than a reference care seeking propensity score for other patients;
weighting the diagnosis risk score by the care seeking propensity score to create a weighted diagnosis risk score;
determining whether the weighted diagnosis risk score indicates a likelihood of the medical diagnosis; and
in response to the determination that the weighted diagnosis risk score indicates a likelihood of the medical diagnosis, transmitting a recommendation for further evaluation to a digital device associated with the patient;
wherein the machine learning model is trained using training data comprising historical claims data, historical clinical data, and historical demographic data, from a population of prior patients, and
wherein the machine learning model is trained to detect correlation between medical diagnosis signals identified from the training data, and a positive result from a screening mechanism for likelihood of the medical diagnosis.

2. The method of claim 1, wherein the prediction target is a claim for a medical procedure within a predetermined amount of time into the future, and wherein the medical procedure is unrelated to the medical diagnosis.

3. The method of claim 2, wherein the prediction target is a claim for an upcoming surgical procedure.

4. The method of claim 1, wherein the medical diagnosis is a mental health diagnosis.

5. The method of claim 4, wherein the screening mechanism is a questionnaire.

6. The method of claim 1, further comprising the steps of,
after the determination that the weighted diagnosis risk score indicates a likelihood of the medical diagnosis, performing the screening mechanism on the patient to obtain a screening result, and
further training of the machine learning model using the screening result, and using at least one of the claims data, the clinical data, and the demographic data.

7. The method of claim 1, further comprising the steps of
obtaining a vocal sample of the patient;
performing vocal sentiment analysis on the vocal sample; and
including vocal sentiment data in the clinical data.

8. The method of claim 1, wherein the demographic data comprises data relating to a location where the patient resides.

9. The method of claim 1, wherein the clinical data comprises comorbidity data.

10. The method of claim 1, wherein the clinical data comprises medication history.

11. The method of claim 1, wherein the clinical data comprises data relating to a history of usage of health services by the patient.

12. The method of claim 1, wherein the clinical data comprises data relating to at least one of patient age, patient insurance coverage, distance between a residence of the patient and medical care, patient access to transportation, patient access to food, and patient access to shelter.

13. The method of claim 1, wherein the clinical data comprises data relating to at least one of health conditions, use of specific medications, medical visits, hospitalizations, laboratory tests, vaccination status, sleep studies, and drug use.

14. The method of claim 1 wherein the clinical data is derived at least in part from an electronic medical record relating to the patient.

15. A system for predicting a medical diagnosis for a patient, independent of interviewing or examining the patient, the system comprising a processor configured to:
receive claims data, clinical data and demographic data relating to the patient;
determine, from the claims data whether a prediction target for the medical diagnosis is present;
in response to a determination that the prediction target is present:
input the prediction target and the clinical data into a machine learning model to predict diagnosis risk, to create a diagnosis risk score;
determine a care seeking propensity score, from the demographic data, wherein the care seeking propensity score is related to whether the patient is a member of a group with a propensity to seek care that is lower than a reference care seeking propensity score for other patients;
weight the diagnosis risk score by the care seeking propensity score to create a weighted diagnosis risk score;
determine whether the weighted diagnosis risk score indicates a likelihood of the medical diagnosis; and
in response to the determination that the weighted diagnosis risk score indicates a likelihood of the medical diagnosis, transmitting a recommendation for further evaluation to a digital device associated with the patient;
wherein the machine learning model is trained using training data comprising historical claims data, historical clinical data, and historical demographic data, from a population of prior patients, and
wherein the machine learning model is trained to detect correlation between medical diagnosis signals identified from the training data, and a positive result from a screening mechanism for likelihood of the medical diagnosis.

16. The system of claim 15, wherein the prediction target is a claim for a medical procedure within a predetermined amount of time into the future, and wherein the medical procedure is unrelated to the medical diagnosis.

17. The system of claim 16, wherein the prediction target is a claim for an upcoming surgical procedure.

18. The system of claim 15, wherein the medical diagnosis is a mental health diagnosis.

19. The system of claim 18, wherein the screening mechanism is a questionnaire.

20. The system of claim 15, wherein the processor is further configured to,
after the determination that the weighted diagnosis risk score indicates a likelihood of the medical diagnosis, perform the screening mechanism on the patient to obtain a screening result, and
further train of the machine learning model using the screening result, and using at least one of the claims data, the clinical data, and the demographic data.
